(19) United States
(12) Patent Application Publication
(10) Pub. No.: US 2020/0221995 A1
(43) Pub. Date: Jul. 16, 2020

(54) SYSTEM AND METHOD FOR DETERMINING THE CONDITION OF HAIR

(71) Applicant: Henkel AG & Co. KGaA, Duesseldorf (DE)

(72) Inventors: CARSTEN MATHIASZYK, Essen (DE); TORSTEN LECHNER, Langenfeld (DE)

(73) Assignee: Henkel AG & Co. KGaA, Duesseldorf (DE)

(21) Appl. No.: 16/638,958

(22) PCT Filed: Aug. 14, 2018
(86) PCT No.: PCT/EP2018/071984

§ 371 Date: Feb. 13, 2020

(30) Foreign Application Priority Data
Aug. 16, 2017 (DE) 10 2017 214 250.2

(51) Int. Cl. A61B 5/00; A61B 5/103; A45D 44/00

(52) U.S. Cl. CPC A61B 5/448; A61B 5/0024; A61B 5/0059; A61B 5/1032; A61B 2562/0204; A61B 5/7221; A45D 44/00; A45D 2044/007; A61B 5/6898

(57) ABSTRACT

A system for determining a hair condition is provided. The system includes a portable sensor device having at least one sensor for detecting at least one sensor value on hairs of a user, and a data processing device. The data processing device is set up to determine whether, in addition to the at least one sensor value, at least one further sensor value from a further sensor device, and is set up to determine the hair condition of the user based on the detected at least one sensor value in a case where it has been determined that only the at least one sensor value is provided, or based on the provided at least one sensor value and the provided at least one further sensor value in a case where it has been determined that the at least one further sensor value is provided.

### CROSS-REFERENCE TO RELATED APPLICATION

This application is a U.S. National-Stage entry under 35 U.S.C. § 371 based on International Application No. PCT/EP2018/071984, filed Aug. 14, 2018, which was published under PCT Article 21(2) and which claims priority to German Application No. 10 2017 214 250.2, filed Aug. 16, 2017, which are all hereby incorporated in their entirety by reference.

### TECHNICAL FIELD

The present disclosure concerns a system and procedure for determining a hair condition and a procedure for determining a recommendation concerning a user's hair.

### BACKGROUND

In many areas of daily life, there has been a trend for some time towards personalized programs that can specifically address individual conditions and needs, for example in a nutrition or health area, but also in a personalized cosmetics area. This can allow a user to find specific cosmetic products and/or receive care instructions that are tailored to the individual needs of his hair, and thus enable a particularly high effectiveness.

When hair is treated with cosmetic products, the effect of the product, e.g. the intensity of a hair colouring, the effectiveness of a care product or the hair reshaping effect of a permanent wave, can strongly depend on the degree of damage to the hair.

Therefore, an assessment of hair damage can be of great importance.

Damage to the hair can be caused by natural or artificial processes. The most important type of damage may be oxidative damage.

For example, the natural processes may have a combined (e.g. simultaneous) effect of UV light and oxygen (O₂) on the hair.

The artificially induced processes may include, for example, the application of hair dye (also referred to as hair colouring, which also includes bleaching), and/or styling or reshaping of the hair (e.g. creating a permanent wave).

Besides desired cosmetic effects, e.g. the lightening of the hair, also severe damage of the hair may occur, e.g. when using oxidizing agents.

In damaged hair, for example, the cysteic acid content may be increased due to oxidation of the amino acids cystine and cysteine, which are very common in hair, to cysteic acid.

The oxidation of cystine/cysteine to cysteic acid can destroy the mechanical stability of the hair and even lead to complete fracture of hair after multiple applications. However, properties of the hair that can already be perceived macroscopically, e.g. tactile properties of the hair, e.g. a surface condition, such as a surface roughness, can be negatively influenced. Damaged hair may, for example, have a higher surface roughness than undamaged hair.

The results of cosmetic treatments may depend on other properties of the treated hair, for example (especially in the case of hair colouring) on the hair colour, hair structure (especially in the case of styling, e.g. perming, straightening, etc.), moisture content (in the case of a care product), etc.

A user can be provided with devices (tools), for example brushes, combs, convenient near infrared (NIR) measuring equipment, microscopes or other devices, by which, for example using a so-called “smart device”, hair damage or another hair condition parameter can be determined. The term “smart device” covers electronic devices that are usually connected to other devices or networks via various wireless protocols such as Bluetooth, NFC, Wi-Fi, 3G, etc. and that are equipped with sensor-based electronic data processing. Examples of “smart devices” are smartphones, phablets, tablets, smart bands, smart mirrors, smart watches or smart key chains. A result of hair damage examination can be displayed on a smartphone, tablet or similar. However, it may be impossible to combine several different results and, for example, evaluate them together.

### SUMMARY

In different exemplary embodiments, a device (e.g. a smartphone, tablet, smart mirror or the like) is provided which may be capable of connecting to all known or future devices/tools (e.g. to determine a hair condition parameter), preferably wirelessly (e.g. via Bluetooth), and thus determining the hair quality (also referred to as hair condition).

For example, brushes, combs, microscopes and/or near infrared devices can be used as devices/tools.

In different exemplary embodiments, the individual examination results can be analysed using software (e.g. an app), for example, individually evaluated. In different exemplary embodiments, the device can be used to compare different results and determine a common hair condition value, so that an improved indication of the hair condition can be provided.

Based on the generated data, the software can recommend suitable hair treatment products and/or procedures for the user. It may also be possible to order the recommended hair treatment products directly online using the software and/or it may be possible to indicate, for example by a display on a screen and/or an announcement via a loudspeaker, which hair treatment products are available.

In different exemplary embodiments, a user can be offered hair treatment product recommendations for hair colouring agents (hair colourings), which can also include bleaching agents, permanent wave, hair care and/or hair styling, according to her/his hair condition.

In addition, a user can be enabled to determine her/his hair condition even more precisely.

A system for determining the condition of hair is provided in different exemplary embodiments. The system may include a portable sensor device with at least one sensor for detecting at least one sensor value on a user's hair and a data processing device.

The data processing device may be set to determine the user's hair condition based on the detected at least one sensor value. However, the sensor device may be modular in design according to different exemplary embodiments. For example, the system comprising of that one sensor device and the data processing device may form a basic version which may be expandable by at least one further sensor device. The data processing device may already be set up in the basic version to determine whether only data (sensor values) are provided for it by the sensor device, or whether at least one further sensor value is also provided for it by a further sensor device.

Depending on the result of the determination, the hair condition may be determined by the data processing device based on that at least one sensor value provided by the sensor device, or based on that at least one sensor value provided by the sensor device in combination with that at least one further sensor value provided by that further sensor device.

In different exemplary embodiments, the hair condition detection system may include an input device.

In different exemplary embodiments, the input device can be used to enter a desired result, such as a desired hair colour, a desired care condition or a desired styling (e.g., curls with a graduated curliness or the like).

In addition, the input device may be used to input empirically determined hair condition parameters, e.g. degree of greying, hair thickness or degree of hair curling, which are determined by looking at or touching the hair.

In different exemplary embodiments, the display and input device may form an integrated unit with the sensor device, for example when using the camera and/or microphone of a smartphone as sensor, or for example when a near infrared spectrometer is equipped with a data processing device and a touch-sensitive display.

In different exemplary embodiments, the hair condition detection system may include an output device, such as a display device.

In different exemplary embodiments, at least one of the sensor devices, the further sensor device and the data processing device may have an acoustic output device in addition to the display device, e.g. a loudspeaker.

In different exemplary embodiments, at least one of the sensor devices, the further sensor device and the data processing device may comprise, in addition to the touch-sensitive screen as an input device or, alternatively, an acoustic input device, e.g. a microphone.

In one case where the sensor device or the further sensor device comprises the microphone, the microphone can in different exemplary embodiments also be used as an input microphone.

In different exemplary embodiments, an alternative or additional conventional input device can be provided as an input device, e.g. a keyboard, a mouse, etc.

In different exemplary embodiments, a standardized and objective evaluation of the treatment result can be made possible by the system, e.g. by the sensor device (possibly supplemented by the further sensor device) in connection with the data processing device. For this purpose, the hair condition of the user after treatment with the hair treatment agent can be determined by the at least one sensor of the sensor device (if necessary additionally by the at least one further sensor of the further sensor device).

The determination of the hair condition can in particular include a determination of a degree of damage to the hair, a determination of a hair colour and/or a determination of a hair moisture. For example, the degree of damage may include a degree of oxidative damage.

In different exemplary embodiments, the system for determining the condition of the hair can be set up such that, by the sensor device and/or the further sensor device, a determination of a degree of oxidative hair damage can be determined exactly by determining a content of cysteic acid. The sensor can be at least one optical sensor which can be set up to take one or more pictures in a fluorescence region and/or in an infrared range (IR range), preferably in a near infrared range (NIR range).

The fluorescence range in different exemplary embodiments can be a wavelength range in which damaged hair emits self-fluorescence and/or a wavelength range in which fluorescent dyes, which are more strongly adsorbed by damaged hair than by undamaged hair, emit fluorescent light.

The IR range, in particular the NIR range, can be a wavelength range in which damaged hair has absorption structures, e.g. in which cysteic acid absorbs light.

Undamaged hair can typically have a cysteic acid content in the range of approx. 0.5% to approx. 1% (by weight). In the presence of damage, for example as a result of multiple ultra-blonding and/or other damage mechanisms, the cysteic acid content can rise to over about 15% (by weight).

In different exemplary embodiments, this property is used to quantify the degree of damage of the hair as cysteic acid content.

In different exemplary embodiments, damaged hair can show a self-fluorescence, which is used to determine the degree of damage by measuring the fluorescence intensity of the hair.

In different exemplary embodiments, the hair can be wetted with a fluorescent dye solution which is better adsorbed by damaged hair than by undamaged hair, whereby the fluorescent dye solution may contain rhodamine B, coumarin and/or fluorescein.

In different exemplary embodiments, the hair can be exposed to UV light (e.g. light in a wavelength range from approx. 315 nm to approx. 380 nm) to determine the fluorescence intensity of the hair. For this purpose, the sensor device or the additional sensor device can be equipped with a UV light source. The UV light source can be a UV LED or another suitable light source, e.g. a conventional UV lamp as used in the authentication of banknotes.

During exposure, fluorescent light emitted by the hair can be registered. Fluorescence intensity can be determined from the registered light. The degree of damage to the hair can then be determined by taking the fluorescence intensity of the hair into account.

Accordingly, the sensor device or the further sensor device in different exemplary embodiments may have an optical sensor which is receptive at least in the fluorescence wavelength range, for example a camera, a photometer, a colorimeter and/or a spectrometer. In different exemplary embodiments, a filter can be placed between the hair and the optical sensor.

In different exemplary embodiments, an infrared (IR) spectrum, especially a near infrared (NIR) spectrum, can be obtained, for example by ATR (Attenuated Total Reflection) (near infrared) spectroscopy. By applying mathematical models, a mathematical model can be created by measuring calibration hair samples which have a cysteic acid content determined using a known analytical method.

In different exemplary embodiments, the model can be used to calculate the cysteic acid content, and thus the hair damage, in an analysis of an IR spectrum recorded on the hair of the user, in particular an NIR spectrum, or at least part of it. An analysis of at least a part of the spectrum and an application of the model can be performed using the data processing device, for example using known smartphones, tablets or the like (with suitable apps).

The sensor device or the additional sensor device may have an IR light source, in particular an NIR light source, for exposing the hair to IR light, in particular NIR light. In this description, the term near infrared (NIR) is used for light with a wavenumber in the range from about 12820 to about 4000 cm-1, and the term infrared (IR) for light with a wavenumber in the range from about 3999 to about 400 cm-1.

The determination of the degree of damage of hair can be carried out according to different exemplary embodiments, either by using the near infrared range, i.e. by irradiating the hair with the near infrared light and spectral analysis of at least a part of the NIR light after it has interacted with the hair, or by using the infrared range, i.e. by irradiating the hair with near infrared light and spectrally analyzing at least a portion of the NIR light after it has interacted with the hair, or using the infrared range, i.e. by irradiating the hair with infrared light and spectrally analyzing at least a portion of the IR light after it has interacted with the hair, or using both the near infrared and (remaining) infrared ranges, i.e. by irradiating the hair with near infrared and infrared light and spectrally analyzing at least a portion of the NIR light and at least a portion of the IR light after it has interacted with the hair.

In different exemplary embodiments, a measured near infrared (NIR) range can have wave numbers from approx. 12820 cm-1 to approx. 4000 cm-1, e.g. from approx. 5022 cm-1 to approx. 4020 cm-1. This wavelength range can show characteristic overtone and combination oscillations of e.g. CH, OH and NH groups.

In different exemplary embodiments, at least part of the near infrared and/or infrared light can have an (infrared) wave number range of approx. 1100 cm-1 to approx. 1000 cm-1, e.g. approx. 1040 cm-1. The relevant absorption bands of the cysteic acid component to be analyzed can be found here.

In different exemplary embodiments, a calibration model can be created for a number of calibration hair samples based on the results of a quantitative computer-aided evaluation (also referred to as chemometric analysis) in combination with values for a cysteic acid content of the respective calibration hair sample obtained for the same calibration hair samples using an independent procedure, e.g. high-pressure liquid chromatography.

Once the calibration model is available, in different exemplary embodiments the concentration of cysteic acid (as a measure of hair damage) for the hair to be measured from the spectra compared to the calibration spectra using the (N)IR spectrum recorded can easily be calculated.

In addition to the direct determination of the cysteic acid content via the characteristic absorption bands of cysteic acid, especially in the range from about 5022 cm-1 to about 4020 cm-1, an indirect determination of the cysteic acid content can also be performed.

There is an inverse correlation between the content of cysteic acid and the content of melanin, which in turn indirectly allows the determination of the content of cysteic acid via the determination of the melanin content. The process of oxidative damage (formation of cysteic acid) during bleaching or dyeing of hair is linked to the degradation of melanin.

By short-wavelength near-infrared spectroscopy in a wave number range from about 12820 to about 7692 cm-1, a wave number range in which cysteic acid does not show any characteristic absorption, reliable calibration models can be generated, which establish a correlation between the short-wavelength near-infrared spectrum and the cysteic acid content.

In different exemplary embodiments, determining the degree of hair damage may involve determining hair damage by interference reflection microscopy.

Very thin hair structures can be examined with the aid of interference reflection microscopy. Interference reflection microscopy is based on the formation of interferences that occur when light is reflected at the upper and lower boundary surfaces of a structure and when reflected light from both boundary surfaces interferes with each other. This results in interference patterns that can be observed and provide information about the thickness of the structure. The resulting interference colours allow structure measurements in the range below about 200 nm. By viewing the interference colours through a light microscope, these structure measurements can be assigned to microscopically recognizable structures.

According to different designs, this can be applied to the cuticle of hair to determine the degree of damage to the hair.

During the exposure of the hair with light, light emitted by the hair can be registered. Based on the registered light, first areas of the hair can be determined which reflect the light with higher interference and therefore appear brighter in an image of the light, and second areas of the hair which reflect the light with lower interference and therefore appear darker.

The hair has a cuticle, cortex and marrow.

If the hair is irradiated with light (e.g. white light), which can be provided by a light source of the sensor device or further sensor device, e.g. a white light LED, a part of the light is reflected at the outer surface of the cuticle and a part is reflected at the boundary surface between cuticle and cortex (especially if the cuticle has moved away from the cortex, which typically corresponds to damage to the hair). The reflected parts interfere and form an interference pattern.

The sensor device or further sensor device may have a camera which may be coupled to an interference reflection microscope of the sensor device or further sensor device. The interference reflection microscope, which may have a magnification factor in a range of approx. 10-1000, e.g. approx. 200-400, may be directed at the hair and image the light reflected from the hair, e.g. from one or more hair fibres, onto a detector of the camera which can register the reflected light as at least one (digital) photo.

The data processing apparatus can determine in different exemplary embodiments for the or each of the photographs the type and/or number of interference patterns of the hair using image analysis software, compare them with a calibration model created in the same way and thus determine a degree of hair damage. For example, in the case of several photographs, the data processing device can average the degrees of damage determined for the photographs.

The interference reflection microscope, the camera, the data processing device and, if necessary, the light source can be implemented in different exemplary embodiments by a smartphone equipped with a microscope lens for smartphones that is also suitable for interference reflection microscopy. In order to ensure stable examination conditions, the hair in different exemplary embodiments can be or will be arranged on a carrier. This can be provided for example by the data processing device (e.g. smartphone) or an attachment for the data processing device.

For example, using a portable electronic device (such as a smartphone, tablet, etc.) with a microscope attachment (such as a Scrona μ peek) in combination with an interference slider (such as offered by Hirox Ltd.), hair can be recorded at 350× magnification.

The area part of the interfering hair structures, i.e. the proportion of bright (damaged) areas in the total area of the hair in the photo, is between about 1% and about 50%, e.g. between about 5% and about 30%.

The data processing device can then deduce the degree of damage from the determined area proportion of bright areas, for example with the help of a table that assigns area parts of areas to degrees of damage.

One type of damage that can be determined by interference reflection microscopy can be mechanical damage, such as that caused by stretching the hair.

In different exemplary embodiments, the external hair damage, for example in the form of surface roughness, can be determined by the sensor device or the further sensor device, which has a sensor for recording acoustic emissions, e.g. a microphone, e.g. by a contact microphone comb.

For the sake of simplicity, the sensor can also be referred to as a microphone. However, unless otherwise stated and suitable for the function described, the sensor may also be an acceleration sensor (which may be suitable to detect accelerations due to acoustic emissions in a certain frequency range) or the like to detect acoustic emissions.

The sensor device or further sensor device can be designed in different exemplary embodiments as a contact microphone comb. The contact microphone comb may have an essentially commercially available hair comb in different exemplary embodiments, to which one or more externally mounted measurement systems for acoustic emissions (i.e. sound emissions; a Korg contact microphone may be used as an exemplary measurement system for acoustic emissions) and/or measurement probes for acoustic emissions are connected. Alternatively, the sensor device or the further sensor device can be designed as a contact microphone brush.

In different exemplary embodiments, the sensor device of the microphone can be used to pick up signals of sound or vibration generated when combing the hair of a user.

The sensor device or the further sensor device in different exemplary embodiments can have one or more measurement systems for acoustic emissions and/or probes, e.g. one or more contact microphones and/or an acceleration sensor. In different exemplary embodiments, the sensor device or further sensor device may also include an internal or external amplifier for amplifying signals measured by the acoustic emission measurement system.

In different exemplary embodiments, an analysis of hair damage can be provided by digitizing information from a microphone (and at least one further sensor) and providing this data.

The information can be provided after processing or for comparison with already recorded examples (reference data). A degree of hair damage can be known for the reference data, so that as a result of the comparison, for example, the degree of hair damage of the most similar reference data can be provided as a result in digital form, e.g. transmitted back.

In different exemplary embodiments, suitable mathematical models of predictive analysis can be used to quantify the cysteic acid content (e.g. by fluorescence analysis and/or by (N)IR spectroscopy) or the (external) degree of damage (e.g. by interference reflection microscopy and/or by acoustic analysis).

In different exemplary embodiments a simple method is provided for use which enables a precise determination of a degree of damage to hair by fluorescence detection and/or by detection of absorption and/or by detection of surface damage to the hair and methods from predictive analytics with the help of fluorescence detection and/or by detection of surface damage to the hair.

In different exemplary embodiments, the determination of hair condition may be suitable for execution using a portable data processing device, also referred to as a mobile data processing device, due to its simple experimental feasibility. For example, a smartphone, an iPad, a tablet or a laptop can be used as a portable data processing device.

In different exemplary embodiments, a method can be provided which enables the use of simple image-analytical methods, e.g. by using a mobile data processing device (e.g. a smartphone), less simple devices (e.g. a UV LED, a white light, NIR and/or IR illumination device, a filter, a portable NIR sensor, a portable (NIR and/or VIS) spectrometer, a microphone comb and/or a microscope) and a predictive analytic method to provide a recommendation regarding the hair of the user, e.g. a hair treatment agent or a hair treatment.

In different exemplary embodiments, the recommended hair treatment agent or treatment may be suitable to achieve a desired effect (e.g., hair colour, hair care condition, hair styling).

In different exemplary embodiments, the use of mathematical models from the field of predictive analytics (e.g. tree ensembles, neural networks or support vector machines) allows a much more exact calculation of the damage (which forms a dependent variable in the models) than would be possible with simple models, e.g. a simple linear regression. The methods can use a large number of input variables in parallel and also map non-linear relationships. In different exemplary embodiments, these models allow, for example, the inclusion of categorical, non-metric input variables, such as a hair color (e.g. blonde, brown, black, etc.) and/or the ethnicity of a hair type (e.g. Caucasian, Asian, African-American), which can have an influence on the measured values. The input variables can be captured in different exemplary embodiments by the sensor device and, if necessary, by the further sensor device, e.g. the hair colour by using a camera (e.g. the smartphone camera), a colorimeter or a colour card, the hair type using the camera.

In different exemplary embodiments, a camera image of the hair can be used to determine not only the hair colour but also a hair structure using image analysis methods. A combination of hair colour and hair structure can be used to determine the ethnic affiliation, e.g. black/curly: African-American, black-smooth: Asian, etc.

According to different design forms, the majority of parameters influencing the measured value can have a hair colour and/or an ethnic affiliation to a type of hair.

According to different exemplary embodiments, predictive analytics can use at least one method from a group of methods, where the group of methods has:

linear or multi-linear regression, polynomial regression, neural network procedures, support vector machine procedures, decision tree procedures (“Decision Trees”, “Random Forest”, “Tree Ensembles”) and other procedures.

By using various sensors such as lenses, gyroscopes and accelerometers, it may be possible to determine a position of the sensor device or further sensor device, e.g. a position in one hand of a person, to determine a suitable cosmetic hair care (e.g. products) and prevent unnecessary hair damage, e.g. hair loss.

In different exemplary embodiments, the sensor device or the further sensor device may have a conductivity sensor for determining hair moisture.

The data transmission of the sensor device to the data processing device and possibly of the further sensor device to the data processing device can take place in different exemplary embodiments by cable or via known radio data transmission methods and/or standards (e.g. Bluetooth, Wi-Fi, NFC, ZigBee, Thread, etc.).

In different exemplary embodiments, the hair condition may have at least one hair condition parameter (e.g. (oxidative) degree of hair damage, degree of extension hair damage, hair color, hair moisture) and each of the at least one sensor value and the at least one further sensor value may be suitable for determining at least one of the hair condition parameters. Each of the sensor values In different exemplary embodiments may be assigned a reliability measure.

In different exemplary embodiments, in a case in which at least one of the at least one sensor value and at least one of the at least one further sensor value are suitable for determining the same hair condition parameter, e.g. the oxidative degree of hair damage, the determination of the hair condition can be carried out by determining the hair condition parameter based on an average value weighted on the basis of the reliability measure numbers, in which case it can be ensured that, in determining the hair condition parameter based on different sensors or measurement methods, the measurement method which has a higher reliability is given greater weighting.

In different exemplary embodiments, only the measurement method (the sensor) with the higher reliability can be evaluated instead of a weighted average value.

In different exemplary embodiments, the sensor device may comprise the NIR sensor, and the further sensor device may comprise the described sensor for determining the degree of hair damage by fluorescent light (i.e. the UV light source and the camera) and/or the contact microphone surface roughness sensor and/or the camera (e.g. for determining the hair colour) and/or the interference reflection microscope and/or the conductivity sensor, and/or a further sensor.

In different exemplary embodiments, the sensor device may comprise the sensor for determining the degree of hair damage by fluorescent light, and the further sensor device may comprise the NIR sensor and/or the contact microphone and/or the camera (e.g. for determining the hair colour) and/or the interference reflection microscope and/or the conductivity sensor, and/or a further sensor.

In different exemplary embodiments, the sensor device may comprise the contact microphone (e.g. designed as a sensor comb), and the further sensor device may comprise the NIR sensor and/or the sensor for determining the degree of hair damage by fluorescent light and/or the camera (e.g. for determining the hair colour) and/or the interference reflection microscope and/or the conductivity sensor, and/or a further sensor.

In different exemplary embodiments, the sensor device may comprise the interference reflection microscope, and the further sensor device may comprise the (N)IR sensor and/or the contact microphone and/or the camera (e.g. for determining the hair colour) and/or the sensor for determining the degree of hair damage by fluorescent light and/or the conductivity sensor, and/or a further sensor.

In different exemplary embodiments, the sensor device may further comprise at least one further sensor, for example one or more of the sensors described which are not part of the second sensor device, or another sensor.

In different exemplary embodiments, a system and a procedure are provided in which hair damage can be measured using various analytical methods. A test result, e.g. a determined degree of hair damage, can be used as an aid for a suitable product recommendation and/or hair treatment procedure recommendation.

In different exemplary embodiments, the system can analyse the hair surface by a (e.g. small) microscope, but also the hair damage (in form of the cysteic acid content) can be determined by the NIR measurement.

In different exemplary embodiments, different analysis methods can be standardized and a hair condition or a hair condition parameter can be determined more easily and quickly.

In different exemplary embodiments, a system for the determination of a hair condition is provided. The system may comprise a portable sensor device having at least one sensor for sensing at least one sensor value on hair of a user, and a data processing device, wherein the data processing device may be arranged to determine whether the data processing device is further provided with at least one further sensor value from a further sensor device in addition to the at least one sensor value, and is arranged to determine the hair condition of the user based on the detected at least one sensor value in a case where it has been determined that only the at least one sensor value is provided, or based on the provided at least one sensor value and the provided at least one further sensor value in a case where it has been determined that the at least one further sensor value is further provided.

In different exemplary embodiments, the system may include at least one additional portable sensor device with at least one further sensor for sensing the at least one further sensor value at the user's hair.

In different exemplary embodiments, hair condition determination may involve determining a degree of hair damage.

In different exemplary embodiments, at least one of the at least one sensor and the at least one further sensor may have an optical sensor for determining a cysteic acid content of the hair and/or for determining a hair colour of the user.

In different exemplary embodiments, the data processing device may be part of a smartphone, tablet or iPad.

In different exemplary embodiments, the sensor device and the data processing device may form an integrated portable device.

In different exemplary embodiments, a method for determining a hair condition is provided. The method may include detecting at least one sensor value on a user's hair by a portable sensor device, providing the at least one sensor value to a data processing device, determining by the data processing device whether the data processing device is further provided with at least one further sensor value from another sensor device, and determining a hair condition of the user by the data processing device based on the detected at least one sensor value in a case where it has been determined that only the at least one sensor value is provided or based on the provided at least one sensor value and the provided at least one further sensor value in a case where it has been determined that the at least one further sensor value is provided.

In different exemplary embodiments, hair condition determination may involve determining a degree of hair damage.

In different exemplary embodiments, determining the degree of hair damage may involve determining a cysteic acid content and/or a surface roughness of the hair.

In different exemplary embodiments, determining a hair condition may involve determining a user's hair moisture.

In different exemplary embodiments, determining a hair condition may involve determining a user's hair colour.

In different exemplary embodiments, the hair condition may have at least one hair condition parameter and each of the at least one sensor value and the at least one further sensor value may be suitable for determining at least one of the hair condition parameters, and each of the sensor values may have a reliability measure associated therewith.

In different exemplary embodiments, in a case where at least one of the at least one sensor value and at least one of the at least one further sensor value are suitable for determining the same hair condition parameter, determining the hair condition can be performed by determining the hair condition parameter based on an average value weighted on the basis of the reliability measures.

In different exemplary embodiments, a method is provided for determining a recommendation regarding hair of a user. The method may include a determination of a hair condition according to different exemplary embodiments and a determination of a recommendation regarding hair of the user, based on the determined hair condition.

In different exemplary embodiments, the user's hair recommendation may include at least one of a hair treatment product recommendation and a hair treatment procedure recommendation.

### BRIEF DESCRIPTION OF THE DRAWINGS

The present disclosure will hereinafter be described in conjunction with the following drawing figures, wherein like numerals denote like elements, and:

FIGS. 1A, 1B and 1C each show a schematic representation of a system for determining a hair condition according to different exemplary embodiments;

FIG. 2 shows a flowchart representing a method for determining a hair condition according to different exemplary embodiments; and

FIG. 3 shows a flowchart representing a method for determining a recommendation concerning hair of a user according to different exemplary embodiments.

### DETAILED DESCRIPTION

The following detailed description refers to the attached drawings which form part of the present application and in which specific embodiments are shown for illustrative purposes in which the present disclosure can be practised. It is understood that other embodiments may be used and structural or logical changes may be made without departing from the scope of protection of the present disclosure. It is understood that the characteristics of the various exemplary embodiments described herein may be combined unless specifically stated otherwise. The following detailed description should therefore not be construed in a restrictive sense and the scope of protection of the present disclosure is defined by the attached claims.

In this description, the terms predictive analytics, big data and data mining are used synonymously.

For a smartphone mentioned herein, unless the context indicates otherwise, this is to be understood as representative for all similar portable data processing devices, i.e. smartphones, tablets, iPads, laptops, etc. The same applies to smartphone cameras and the like.

A hair colorant is a changing a hair colour. The hair colorant can be either a coloration to create a hair color (e.g. black, brown, red), or a bleaching agent to lighten a hair colour.

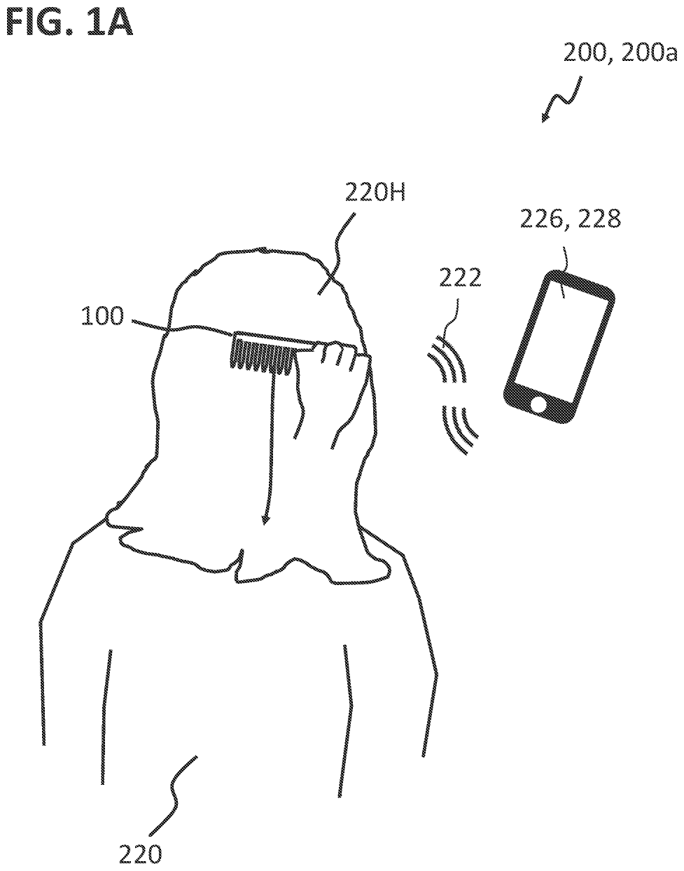

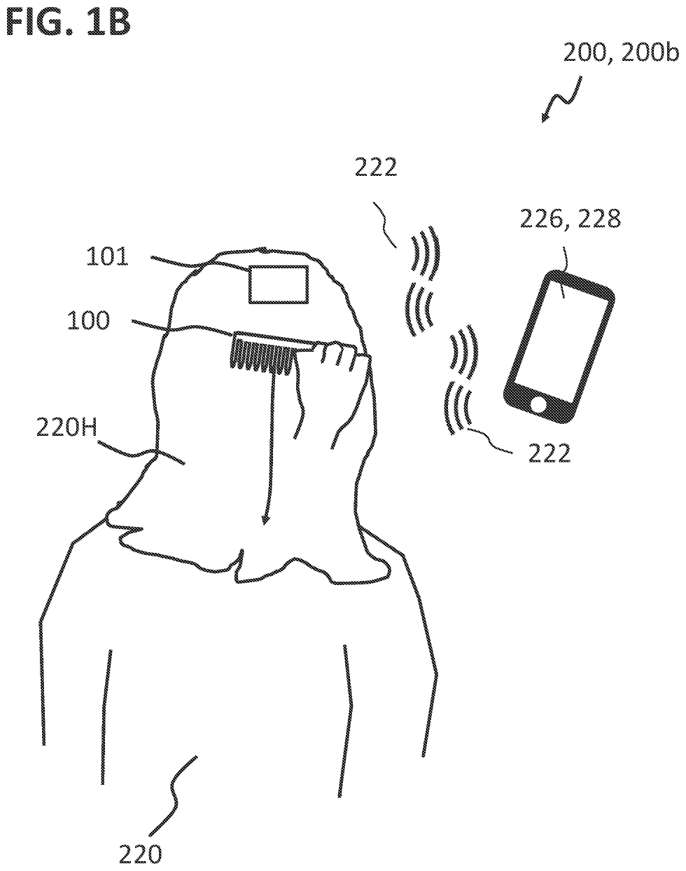

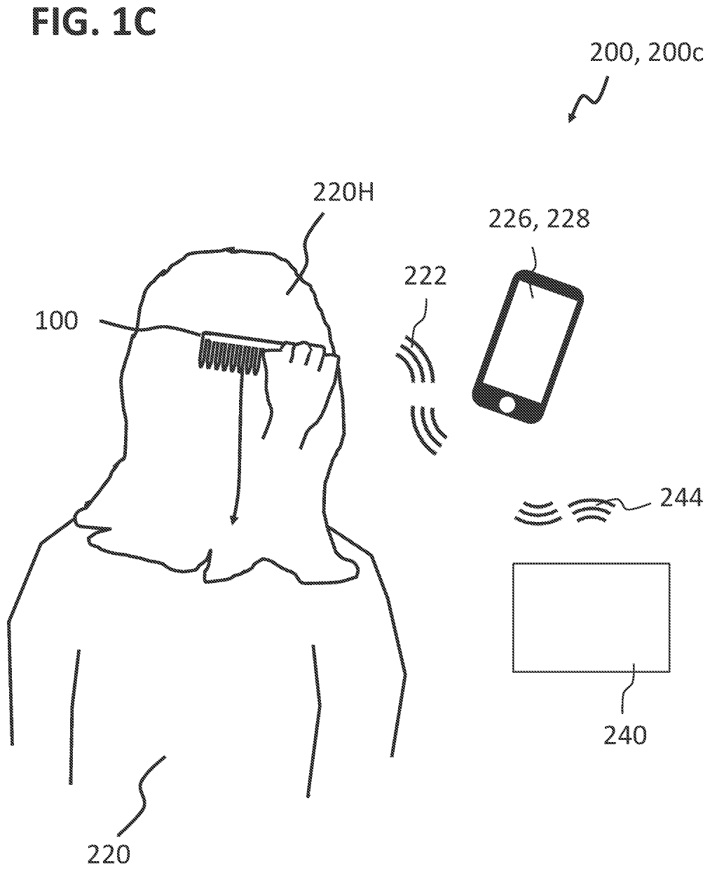

FIGS. 1A, 1B and 1C each show a schematic representation of a system to determine a hair condition according to different exemplary embodiments.

Different exemplary embodiments of the system 200 (e.g. system 200a, 200b, 200c) for determining a hair condition are marked with trailing letters.

In different exemplary embodiments, the System 200 may comprise a portable sensor device 100 with at least one sensor for detecting at least one sensor value on hair 220H of a user 220.

In different exemplary embodiments, the System 200 may include a data processing device 226.

In different exemplary embodiments, the data processing device 226 of system 200b may be set up to determine whether at least one further sensor value is provided to the data processing device 226 by a further sensor device 101 (see FIG. 1B) in addition to the at least one sensor value (from the sensor device 100). The data processing device 226 may be set up to determine the hair condition of user 220 based on the detected at least one sensor value in a case where it has been determined that only the at least one sensor value is provided, or to determine based on the provided at least one sensor value and the provided at least one further sensor value in a case where it has been determined that the at least one further sensor value is further provided.

In other words, the System 200 in different exemplary embodiments can be provided as a modular system in which the sensor device 100 may be sufficient to detect the hair condition of the user 220, but also one or more further sensor device(s) 101, each of which may have one or more further sensors, in the determination of the hair condition, for example in order to determine further hair condition parameters in addition to one or more hair condition parameters by which the hair condition can be described and which can be determined by the sensor device 100, and/or in order to improve an accuracy of the hair condition parameter determined by the sensor device 100.

In addition, the modular design of the System 200 allows the user 220 to purchase a relatively inexpensive basic system (such as the system 200a as shown in FIG. 1A) and later expand it with one or more additional portable sensor device(s) 101 (such as the system 200b as shown in FIG. 1B).

In different exemplary embodiments, the hair condition detection system may include an output device 228, e.g. a display device (e.g. a screen and/or microphone of a smartphone).

In different exemplary embodiments, the hair condition detection system may include an input device, such as a touchscreen 228 of a smartphone.

In different exemplary embodiments, hair condition determination, as described above, may include determining a degree of damage to hair 220H (for example, as oxidative hair damage degree and/or as mechanical (e.g., stretch) hair damage degree), a hair colour, hair moisture, or other or additional hair condition parameters.

In different exemplary embodiments, the at least one sensor (of the sensor device 100) may comprise one or more of the sensors described above, e.g. an (N)IR sensor, a sensor described above for determining the degree of hair damage by fluorescent light (i.e. with UV light source camera), a contact microphone (for example for determining the surface roughness), a camera (e.g. for determining the hair colour), an interference reflection microscope, a conductivity sensor and/or another sensor.

In different exemplary embodiments, the at least one further sensor (of the further sensor device 101) may comprise one or more of the sensors described above, e.g. an NIR sensor, a sensor described above for determining the degree of hair damage by fluorescent light (i.e. with UV light source camera), a contact microphone surface roughness sensor, a camera (e.g. for determining the hair colour), an interference reflection microscope, a conductivity sensor and/or a further sensor.

The sensor or the further sensor for determining a cysteic acid content of the hair may have, as described above, in different exemplary embodiments, a camera and/or a spectrometer for detecting light in a wavelength range of visible light, the light being emitted from the hair as fluorescent light when irradiated with UV light.

In different exemplary embodiments, a single sensor can be set up on the sensor or the further sensor in such a way that only the fluorescent light can be evaluated, for example in a case where at least one filter is set up between the hair and the optical sensor, which or at least one of which allows only or mainly a wavelength range of the fluorescent light to pass through.

If it is intended in such a case to additionally detect the hair colour by the sensor device 100 or 101, the filter can be set up to be removable, for example as a filter attachment for a smartphone camera, so that the detection of the fluorescent light and a detection of visible light in several wavelength ranges (e.g. R, G, B) for determining the hair colour can take place sequentially. Alternatively, the filter can have several regions, for example a bandpass region for the fluorescent light, possibly a bandpass region outside the fluorescent light wavelength range and/or an essentially unfiltered region. Alternatively, the at least one sensor or further sensors can have a multiplicity of sensors, whereby at least one of the sensors is used to detect the fluorescence light, and another of the sensors is set up to determine the hair colour, for example as a camera, e.g. a camera of a smartphone, tablet, etc., or e.g. as a spectrometer.

In different exemplary embodiments, a single sensor can be set up for the sensor or the other sensor in such a way that both the fluorescent light can be evaluated and the user's hair colour can be determined, for example when designing the sensor of the sensor or the further sensor as a spectrometer.

The sensor or the further sensor for determining a cysteic acid content of the hair can, as described above, have an (N)IR camera and/or an (N)IR spectrometer for the detection of light in a wavelength range in which the cysteic acid absorbs light in different exemplary embodiments.

If, in such a case, it is intended to additionally detect the hair colour by the sensor device 100 or 101, the sensor device 100 or 101 may comprise a further sensor for detecting the visible light for determining the hair colour.

In different exemplary embodiments, the at least one sensor of the sensor device 100 and the at least one further sensor of the further sensor device 101 can be different sensors.

In different exemplary embodiments, the data processing device 226 may be part of a smartphone, tablet or laptop, as exemplified in FIGS. 1A, 1B and 1C.

In different exemplary embodiments, the sensor device 100 and the data processing device 226 may form an integrated portable device, for example, in a case where the sensor is part of the data processing device 226, e.g., when using a camera and/or microphone of a smartphone as the sensor.

In different exemplary embodiments, the registered light and/or the registered noise or, if applicable, another or further recorded sensor value as signal 222, e.g. as raw data and/or in processed form, e.g. as digital photo or another quantification of the registered light, can be transmitted as audio file, Fourier transformation or similar to the data processing device 226.

Transmission may be, in different exemplary embodiments, in a known manner, for example by a data cable, wireless data transmission (e.g. Bluetooth or Near Field Communication (NFC)), or transmission may be within a device if the sensor device 100 forms the integrated device with the data processing device 226 as described above (e.g. when using the camera and/or microphone of a smartphone, tablet, laptop or the like as a camera).

In different exemplary embodiments, the sensor device 100 may be designed as a comb or brush, as shown in FIGS. 1A, 1B and 1C as examples. In other words, the at least one sensor can be integrated into a comb- or brush-shaped body.

In different exemplary embodiments, the microphone in particular can be integrated into the comb- or brush-shaped body, for example as described above. In different exemplary embodiments, the microphone can be attached to/into teeth or to/into the bristles of the comb- or brush-shaped body.

In different exemplary embodiments one or more sensors can be alternatively or additionally integrated into the comb- or brush-shaped body.

In different exemplary embodiments, the sensor device 100 or the further sensor device 101 can have a body that is essentially arbitrarily shaped, if practical.

In different exemplary embodiments, the sensor device 100 and/or the further sensor device 101 and the data processing device 226 can form an integrated device.

In different exemplary embodiments, the sensor device 100 or the further sensor device 101 and/or the data processing device 226 may have a device for wireless data transmission, for example for data transmission via Wi-Fi, Bluetooth, Thread, ZigBee, NFC or similar, e.g. as described above.

For receiving and further processing the signals/data, the data processing device 226, in different exemplary embodiments, may be equipped with appropriate software, such as an app, as described above. A single software/app may be provided to determine the condition of hair based on the sensor values from sensor device 100 and to determine the condition of hair based on the sensor values from sensor device and further sensor device 101.

In different exemplary embodiments, the data processing device 226 can be used to determine a cysteic acid content based on the light intensity determined (e.g. fluorescent light or (N)IR light). As described above, mathematical models from the field of predictive analytics can be used to determine a relationship between the (standardized) light intensity and an associated cysteic acid content (and thus a degree of damage to the hair).

In different exemplary embodiments, for example, a relationship between cysteic acid content and light intensity or corresponding data values, e.g. in the form of assigned data pairs, can be included as independent parameters in the model, which were determined or mathematically modelled by measuring standard hair samples which have a cysteic acid content determined using known complex methods.

Accordingly, in different exemplary embodiments with other sensor values can be operated to determine the degree of damage of the hair and/or the hair colour.

In different exemplary embodiments, the degree of damage of the hair can be determined in a categorial scale (e.g. light, medium, severe).

In different exemplary embodiments, the degree of damage can be determined on a metric scale (e.g. percentage of cysteic acid content, percentage of areas with higher interference, etc.).

The data processing device 226 may, as described above, comprise a mobile data processing device, for example a smartphone, a tablet or a laptop, in particular in a case where the portable sensor device 100 forms the integrated device with the data processing device 226.

In different exemplary embodiments, the data processing device 226 may be of a different type, e.g. a desktop computer integrated into a smart mirror, or any other data processing device 226 capable of storing and providing the data and performing the predictive analytics method, e.g. each data processing device 226 with sufficiently large data memory and sufficiently powerful processor.

In different exemplary embodiments, instead of the predictive-analytical method, other, e.g. simpler, methods can be used to determine the condition of the hair, e.g. the degree of damage, the colour of the hair and/or the moisture of the hair. For example, classification regulations may be mentioned.

In different exemplary embodiments, as represented in FIG. 1C, the data processing device 226 of the system 200c may be arranged to indirectly determine the condition of hair, e.g. the degree of hair damage and/or the colour of hair, and/or a recommendation based on the condition of hair, for example by transmitting a signal 244 (e.g. the sensor raw data and/or partially evaluated sensor data and/or the determined hair condition) to an external data processing device 240, for example to a cloud processor architecture (short: cloud), and by receiving a result from the external data processing device 240 (e.g. the cloud).

In different exemplary embodiments, the device for providing the hair treatment agent may be designed as a learning system, for example by the user and/or further users providing the hair condition prior to an application of the hair treatment agent and the hair condition after a treatment of the hair with the hair treatment agent of the data processing device 226 (for example by the cloud).

In different exemplary embodiments, a determination of the hair condition after treatment with the hair treatment agent can be used to provide the hair condition after treatment by the System 200 for determining the hair condition.

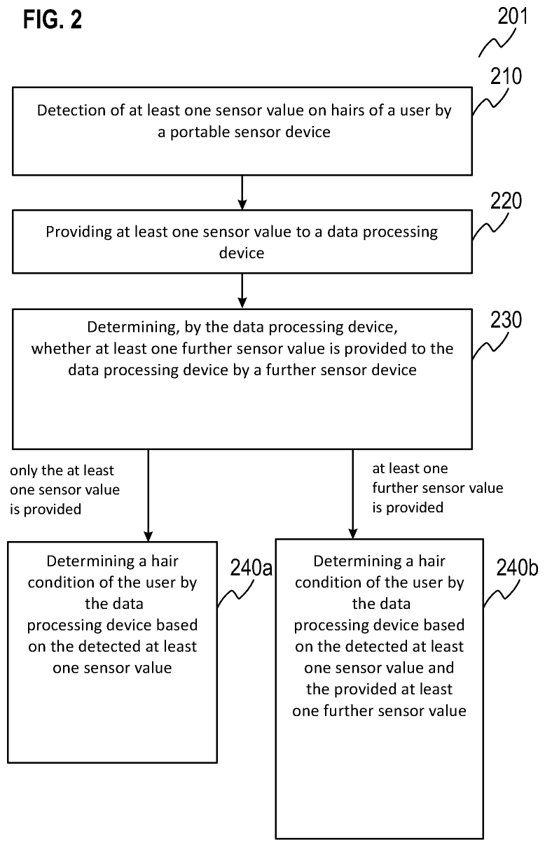

FIG. 2 shows a flowchart 201, which shows a procedure to determine a hair condition according to different exemplary embodiments. To perform the procedure, a device according to different exemplary embodiments, as described above, can be used.

The method may include detecting at least one sensor value on hairs of a user by a portable sensor device (at 210), providing the at least one sensor value to a data processing device (at 220), determining by the data processing device whether the data processing device is further provided with at least one further sensor value from a further sensor device (at 230), and either determining a hair condition of the user by the data processing device based on the detected at least one sensor value (at 240a, in a case where only the at least one sensor value is provided) or determining a hair condition of the user by the data processing device based on the detected at least one sensor value and the provided at least one further sensor value (at 240b, in a case where the at least one further sensor value is provided).

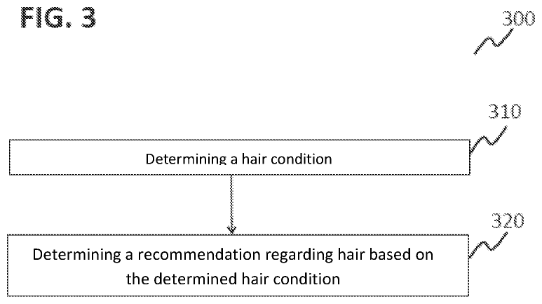

FIG. 3 shows a flowchart 300 representing a method for determining a recommendation regarding hair of a user according to different exemplary embodiments.

The method may include determining a hair condition according to different exemplary embodiments (at 310), and determining a hair recommendation based on the determined hair condition (at 320).

The recommendation, in different exemplary embodiments, can be a product recommendation and/or a hair treatment recommendation.

In different exemplary embodiments, the determination of the hair condition may include at least a determination of the degree of damage of the hair.

Further advantageous designs of the method result from the description of the device and vice versa.

While at least one exemplary embodiment has been presented in the foregoing detailed description, it should be appreciated that a vast number of variations exist. It should also be appreciated that the exemplary embodiment or exemplary embodiments are only examples, and are not intended to limit the scope, applicability, or configuration of the various embodiments in any way. Rather, the foregoing detailed description will provide those skilled in the art with a convenient road map for implementing an exemplary embodiment as contemplated herein. It being understood that various changes may be made in the function and arrangement of elements described in an exemplary embodiment without departing from the scope of the various embodiments as set forth in the appended claims.

1. A system for determining a hair condition comprising:
a portable sensor device having at least one sensor for detecting at least one sensor value on hairs of a user; and
a data processing device set up to determine whether, in addition to the at least one sensor value, at least one further sensor value is provided to the data processing device by a further sensor device, and being set up to determine the hair condition of the user based on the detected at least one sensor value in a case where the data processing device has determined that only the at least one sensor value is provided, or based on the provided at least one sensor value and the provided at least one further sensor value in a case where the data processing device has determined that the at least one further sensor value is provided.

2. A system according to claim 1, further comprising:
the further sensor device is further defined as a further portable sensor device having at least one further sensor for detecting the at least one further sensor value at the hairs of the user.

3. A system according to claim 1,
wherein the data processing device is set up to determine the user's hair condition by determining a degree of damage of the hair.

4. A system according to claim 1,
wherein the at least one sensor and/or the at least one further sensor comprises an optical sensor for determining a cysteic acid content of the hair and/or for determining a hair colour of the user.

5. A system according to claim 1,
wherein the at least one sensor or the at least one further sensor comprises a microphone for determining a surface roughness of the hair.

6. A system according to claim 1,
wherein the data processing device is part of a smartphone, tablet or iPad.

7. A system according to claim 1,
wherein the first portable sensor device and the data processing device form an integrated portable device.

8. A method for determining a condition of hair, comprising:
detecting at least one sensor value on hairs of a user by a portable sensor device;
providing said at least one sensor value to a data processing device;
determining, by the data processing device, whether the data processing device is further provided with at least one further sensor value from a further sensor device; and
determining a user's hair condition by the data processing device based on the detected at least one sensor value in a case where the data processing device has determined that only the at least one sensor value is provided, or based on the provided at least one sensor value and the provided at least one further sensor value in a case where the data processing device has determined that further the at least one further sensor value is provided.

9. A method according to claim 8,
wherein the determining of the user's hair condition comprises determining a degree of damage of the hair.

10. A method according to claim 8,
wherein the determining of the user's hair condition comprises determining a hair moisture of the user.

11. A method according to claim 8,
wherein the hair condition comprises at least one hair condition parameter and each of the at least one sensor value and the at least one further sensor value is suitable for determining at least one of the hair condition parameters, and wherein each of the at least one sensor value and the at least one further sensor value has a reliability measure associated therewith.

12. A method according to claim 11,
wherein, in a case where the at least one sensor value and the at least one further sensor value are suitable for determining the same hair condition parameter, the determining of the user's hair condition includes determining the hair condition parameter based on an average value weighted based on the reliability measures.

13. A method for determining a recommendation regarding hair of a user, comprising:
determining a hair condition according to claim 8; and
determining a recommendation regarding the hair of the user based on the determined hair condition.

14. A method according to claim 13,
wherein the hair recommendation of a user comprises at least one of a hair treatment product recommendation and a hair treatment method recommendation.

15. A method according to claim 13,
wherein the user is enabled to order the recommended hair treatment product online and/or the user is informed where the recommended hair treatment product is available.
